(19) United States
(12) Patent Application Publication
(10) Pub. No.: US 2019/0066836 A1
(43) Pub. Date: Feb. 28, 2019

(54) METHODS AND SYSTEMS FOR DETERMINING COLLECTABLE TIME OF A MEDICAL PROFESSIONAL USING A FACILITY

(71) Applicant: LeanTaas, Santa Clara, CA (US)

(72) Inventors: Zetong LI, Menlo Park, CA (US); Ashley WALSH, San Jose, CA (US)

(21) Appl. No.: 16/108,051

(22) Filed: Aug. 21, 2018

Related U.S. Application Data
(60) Provisional application No. 62/548,977, filed on Aug. 23, 2017.

(51) Int. Cl. G16H 40/20

(52) U.S. Cl. CPC G16H 40/20

(57) ABSTRACT

Method and system for determining appropriate block allocation reduction of medical professional using facility are disclosed. Method includes accessing facility usage data of facility by medical professional for predefined time. The facility usage data comprises allocated time of medical professional for using facility, actual usage time of facility from allocated time, idle time of facility from allocated time and released time from allocated time of facility. The method includes provisioning of at least one UI on display screen of electronic device for receiving user inputs. The user inputs comprise first threshold associated with idle time and second threshold associated with released time. The method includes determining collectable idle time based on comparison of idle time and first threshold. The method includes determining collectable released time based on comparison of released time and second threshold. The method further includes calculating collectable time by aggregating collectable idle time and collectable released time.

### TECHNICAL FIELD

Embodiments of the disclosure relate generally to scheduling and allocation of resources and, more particularly to, methods and systems for allocation of facilities such as operating rooms of a hospital to medical professionals.

### BACKGROUND

Facilities such as operating rooms (ORs) or diagnostic rooms are the important resources of a hospital, as they provide a considerable revenue for the hospital. Particularly, operating room allocation and planning is one of the most important strategic decision that OR managers face since optimizing OR utilization ensures high surgical output, increased revenue for the hospital, catering to a large number of patients and greater patient satisfaction. However, on the contrary, inefficient OR allocation can result in cancellations of appointments and long patient waiting lists.

An OR is generally assigned to a specific surgeon or a group of surgeons of a medical department, for example, cardiology department for a specific time (also referred to as ‘a block’) so as to schedule surgeries in the block assigned to them. The surgeon or the medical department is assigned one or more blocks each week in which surgery cases of the surgeon or the medical department could be scheduled.

A hospital administrator, such as an OR manager manually allocates blocks to surgeons based on historical block utilization data and simple rules (such as surgeon seniority) as the basis of block allocation decisions. However, this approach is not very effective since it does not take caseload variability into account on the part of the surgeons. For example, low block utilization by one surgeon may be due to the surgeon being on vacation, or because that surgeon's case length tends to be a few hours shorter than the block length. Without comprehensive and easy-to-understand data, the block allocation becomes more of a political play instead of a data-driven decision process. Consequently, many precious hours of block time go unused.

In the light of the above discussion, there is a need for methods and systems to determine the reasonable allocation of blocks of a surgeon or medical department along with a unique set of visualizations that captures all necessary information to provide supporting evidence for determining a part of time in the block that is avoidable idle time.

### SUMMARY

Various embodiments of the present disclosure provide methods and systems for determining the reasonable allocation of OR time of a medical professional or a medical department that has been assigned recurring OR time.

In an embodiment, a method is disclosed. The method includes accessing a facility usage data of a facility by a medical professional for a predefined time. The facility usage data comprises an allocated time of the medical professional for using the facility, an actual usage time of the facility from the allocated time, an idle time of the facility from the allocated time, and a released time from the allocated time of the medical professional. The method includes provisioning of at least one User Interface (UI) on a display screen of an electronic device associated with a user for receiving user inputs. The user inputs comprise a first threshold associated with the idle time and a second threshold associated with the released time. The method includes determine a collectable idle time based on a comparison of the idle time and the first threshold. The method includes determine a collectable released time based on a comparison of the total released time and the second threshold. The method further includes calculating a collectable time associated with the medical professional by aggregating the collectable idle time and the collectable released time.

In another embodiment, a server is disclosed. The server comprises a memory to store instructions and a processor to execute the stored instructions in the memory and thereby cause the system to access a facility usage data of a facility by a medical professional for a predefined time. The facility usage data comprises an allocated time of the medical professional for using the facility, an actual usage time of the facility from the allocated time, an idle time of the facility from the allocated time, and a released time from the allocated time of the medical professional. The system is further caused to provision at least one User Interface (UI) on a display screen of an electronic device associated with a user for receiving user inputs. The user inputs comprise a first threshold associated with the idle time and a second threshold associated with the released time. The system is further caused to determine a collectable idle time based on a comparison of the idle time and the first threshold. The system is further caused to determine a collectable released time based on a comparison of the released time and the second threshold. The system is further caused to calculate a collectable time associated with the medical professional by aggregating the collectable idle time and the collectable released time.

In yet another embodiment, a method is disclosed. The method includes accessing a facility usage data of a facility by a medical department comprising one or more medical professionals for a predefined time. The facility usage data comprises an allocated time of the medical department for using the facility, an actual usage time of the facility from the allocated time, an idle time of the facility from the allocated time, and a released time from the allocated time of the medical department. The method includes provisioning of at least one User Interface (UI) on a display screen of an electronic device associated with a user for receiving user inputs. The user inputs comprise a first threshold associated with the idle time and a second threshold associated with the released time. The method includes determine a collectable idle time based on a comparison of the idle time and the first threshold. The method includes determine a collectable released time based on a comparison of the released time and the second threshold. The method further includes calculating a collectable time associated with the medical department by aggregating the collectable idle time and the collectable released time.

### BRIEF DESCRIPTION OF THE FIGURES

For a more complete understanding of example embodiments of the present technology, reference is now made to the following descriptions taken in connection with the accompanying drawings in which:

FIG. 1 is an illustration of an environment, where at least some example embodiments can be practiced;

FIG. 2 is a graphical representation depicting distribution of block usage data, in accordance with an example embodiment;

FIG. 3 is a block diagram representation of a system for determining collectable time of a medical professional using a facility, in accordance with an example embodiment;

FIG. 4 shows an example representation of a table displaying collectable percentage and collectable blocks of each medical department, in accordance with an example embodiment;

FIG. 5A shows an example representation of a UI depicting data visualization for a plurality of medical professionals using the facility, in accordance with an example embodiment;

FIG. 5B shows an example representation of an overlay depicting user inputs provided by an administrator for determining collectable time of the plurality of medical professionals using the facility, in accordance with an example embodiment;

FIG. 5C shows an example representation of a UI displaying supporting evidence for the collectable blocks of a medical professional, in accordance with an example embodiment;

FIG. 5D shows a representation of a block decision letter sent to a medical professional, in accordance with an example embodiment;

FIG. 6A is a flowchart illustrating a method for determining collectable time of the medical professional using the facility, in accordance with an example embodiment;

FIG. 6B is a flowchart illustrating a method for determining collectable time of a medical department using a plurality of facilities, in accordance with another example embodiment;

FIG. 7 is a block diagram of a user device, in accordance with an example embodiment; and

FIG. 8 is a block diagram of the server system of FIG. 1, in accordance with an example embodiment.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. In other instances, systems and methods are shown in block diagram form only in order to avoid obscuring the present disclosure.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

### OVERVIEW

Various example embodiments of the present disclosure provide methods and systems for determining collectable time of a medical professional or a medical department using one or more facilities, such as operating rooms of a hospital.

A medical professional is usually assigned a certain amount of time (allocated time) each week for performing medical procedures at a facility, for example, operation room in a hospital. Accordingly, the medical professional may schedule surgeries in the allocated time. In some cases, medical professionals tend to obtain and retain as much time as they can so as to access the operations rooms only at select time frames at their convenience. However, this may result in ineffective utilization of the allocated time at the operation room by the medical professional. Moreover, techniques are employed for accessing historical block utilization data and a percentage of allocated time used by the medical professional/medical department of the allocated time at the OR. However, such measures can hardly provide valid reasons supporting the argument of reduction of allocated time in an OR for a medical professional. This impedes efficient utilization of the operation rooms by the medical professional for scheduling the surgeries. Accordingly, there is a need for an efficient method to determine an idle time of the operating rooms and optimize scheduling of the operating rooms to the medical professionals based on a usage data of the facility by each medical professional.

In at least one example embodiment, a facility management application can be used to determine a collectable time from the allocated time for the medical professional using the operation room (hereinafter referred to as ‘a facility’). The collectable time refers to a potential actionable reduction of the allocated time.

The term “collectable time” implies avoidable idle time of the facility that may be allocated to another medical professional or a medical department for efficient utilization of the facility. It shall be noted that the term ‘operating room’ refers to a facility where one or more medical professionals perform a medical procedure on a patient, and the terms ‘operating room’ and ‘facility’ have been interchangeably used throughout this description.

An administrator, such as, a hospital administrator or an operating room scheduler allocates one or more blocks in a predefined time for each medical professional based on caseload or demand of the medical professional. It shall be noted that each of the one or more blocks scheduled for the medical professional may be for a specific time (also referred to as ‘block length’) and an aggregation of the block lengths of the one or more blocks constitute the allocated time of the medical professional for using the facility. In at least one example embodiment, a facility management application is configured to facilitate management of the facility. The term ‘management’ of the facility implies scheduling of blocks and determining collectable blocks for each medical professional.

The facility management application accesses the electronic medical records (EMR) maintained at a hospital to obtain information of the facility such as, a block schedule data, a block usage information, an operating room case data, details of medical professionals performing a medical procedure, time duration of the medical procedure and delays in initiating a medical procedure. The information is used to determine a facility usage data of the facility by the medical professionals. The facility usage data of a medical professional includes information such as, an allocated time of the medical professional for using the facility, an actual usage time of the facility from the allocated time, an idle time of the facility from the allocated time, and a released time of the facility from the allocated time. The actual usage time implies well-utilized time of the medical professional for performing medical procedures at the facility. The idle time indicates unused time of the facility despite being allocated to the medical professional for performing medical procedures, and the released time of the medical professional refers to a part of the allocated time which was released by the medical professional.

Further, the facility management application provides an option for the administrator to provide user inputs. The user inputs comprise a first threshold and a second threshold that are used for determining collectable time associated with the medical professional. The first threshold is compared with the idle time to determine a collectable idle time. In at least one example embodiment, the idle time above the first threshold constitutes the collectable idle time. The second threshold is compared with the released time to determine a collectable released time. In an embodiment, the released time above the second threshold constitutes the collectable released time from the allocated time of the medical professional. The collectable idle time and the collectable released time are aggregated to determine the collectable time of the medical professional using the facility. It shall be noted that the idle time below the first threshold and the released time below the second threshold constitute non-collectable time and are not used to determine a number of collectable blocks from the medical professional using the facility. Further, the collectable time and the non-collectable time may vary based on the user inputs provided by the administrator.

In at least one example embodiment, the facility management application provides a professional interface that can be accessed by the medical professional for viewing block usage data. The professional interface displays the block usage data of the facility by the medical professional for performing medical procedures over the allocated time for a predefined time, say, past quarter of a year and a collectable time of the medical professional based on the block usage data. Additionally, the professional interface displays a total number of collectable blocks of the medical professional based on the collectable time and the allocated time. Further, the professional interface also displays one or more data visualizations that depict the block usage data by the medical professional for the predefined time and is supplemented with supporting evidence, such as a block usage pattern by the medical professional, a total number of allocated blocks based on the allocated time of the medical professional, the total number of collectable blocks and the block usage data. The block usage data depicts an actual usage time of the facility from the allocated time, an idle time of the facility below the first threshold from the allocated time, an idle time of the facility above the first threshold from the allocated time, a released time below the second threshold from the allocated time of the medical professional and a released time above the second threshold from the allocated time of the medical professional. The non-collectable time is an aggregation of the actual usage time, the idle time below the first threshold and the release time within the second threshold. The medical professionals can view their collectable time that is based on the block usage data along with supporting evidence that provides a proof of the collectable time.

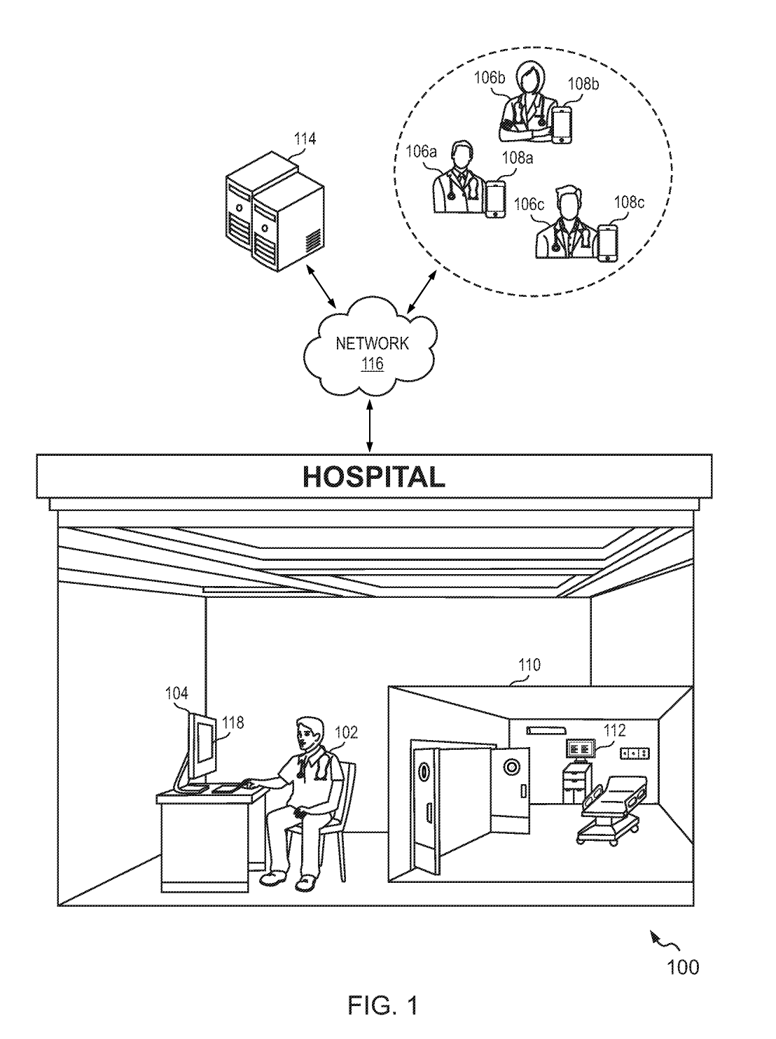

Various embodiments of the facility management application are explained with reference to FIGS. 1-8.

FIG. 1 is an illustration of an environment 100 where at least some example embodiments may be practiced. The environment 100 is depicted as a hospital where a wireless communication network (e.g., a network 116) connects entities such as, a user 102 (also called an OR manager 102), a plurality of medical professionals 106a, 106b and 106c, a facility 110 (hereinafter referred to as an ‘operating room 110’) and a server 114. Examples of the user 102 include, but are not limited to, hospital administrators, nurse managers and operating room (OR) schedulers. It will be appreciated by those skilled in the art that one or more users and one or more facilities may be present in the environment 100, a number of such constituents are shown in FIG. 1 only for example purposes.

The user 102 is depicted to be associated with an electronic device 104 (hereinafter referred to as a ‘user device 104’). In at least one example embodiment, the user device 104 is equipped with a facility management application 118 for managing the facility 110 and optimizing the utilization of the facility 110 by the medical professionals 106a, 106b and 106c. The user device 104 may be any communication device having hardware components for enabling a User Interface (UI) of the facility management application 118 to be presented on the user device 104. The user device 104 may be capable of being connected to a wireless communication network (such as the network 116). Examples of the user device 104 include a mobile phone, a smart telephone, a computer, a laptop, a PDA (Personal Digital Assistant), a Mobile Internet Device (MID), a tablet computer, an Ultra-Mobile personal computer (UMPC), a phablet computer, a handheld personal computer and the like. The user 102 accesses the facility management application 118 for allocating one or more blocks in a predefined time to each medical professional of the plurality of medical professionals 106a, 106b and 106c. The allocation of the one or more blocks for a medical professional (e.g., the medical professional 106a) may either be based on caseload or demand of the medical professional 106a. It shall be noted that each block is of a block length as specified by the user while allocating the block for a medical professional (e.g., the medical professional 106a).

The block length of the one or more blocks may be fixed or variable depending on the caseload and category of medical procedure being performed in a block by the medical professionals 106a, 106b, 106c. The block length is a measure of time allocated in a block for a medical professional and the allocated time is the cumulative of the block length of the one or more blocks allocated for the medical professional (e.g., the medical professional 106a). In an example, the medical professional 106b has been allocated 4 blocks per week, for example, Monday at 10:00 AM, Wednesday at 2 PM, Thursday at 1 PM and Saturday at 11 AM for performing medical procedures at the facility 110. The blocks on Monday, Wednesday, Thursday and Saturday are of block length 2 hours, 3 hours, 2 hours and 4 hours, respectively. The aggregation of the block lengths of the blocks assigned to the medical professional 106b in the week sums up to give the allocated time of 11 hours/week (4 blocks/week) or 44 hours/month (16 blocks/month) or 132 hours/quarter (48 blocks/quarter) for the medical professional 106b. In another example scenario, where the block length for the medical professionals 106a, 106b and 106c are fixed, assuming the facility 110 can be utilized by the medical professionals 106a, 106b and 106c for 84 hours/week and the block length is fixed at 2 hours, a total number of blocks that can be allocated to the plurality of medical professionals 106a, 106b and 106c will account to 42 blocks (total number of blocks/block length=84/2). These 42 blocks are allocated to the medical professionals 106a, 106b and 106c based on the caseload or demand of each of the medical professionals 106a, 106b and 106c. If the blocks (42 blocks/week) are equally allocated between the medical professionals 106a, 106b and 106c, each medical professional will get 14 blocks/week or 28 hours/week for using the facility 110. The facility 110 may be accessed by the medical professionals 106a, 106b and 106c for performing medical procedures based on the allocated time for each medical professional (e.g., the medical professional 106a). The facility management application 118 is configured to display a facility scheduler table that depicts a schedule for a week, for example, a day of the week, a start time of a block, an end time of the block, details of medical professionals authorized to access the facility 110 for the block and allocated time for each of the medical professionals.

The plurality of medical professionals 106a, 106b and 106c are depicted to be associated with one or more devices; for example, the medical professional 106a is associated with the device 108a, the medical professional 106b is associated with the device 108b and the medical professional 106c is associated with the device 108c. The devices 108a, 108b and 108c are equipped with an instance of the facility management application 118. The facility management application 118 facilitates a professional interface for the plurality of medical professionals 106a, 106b and 106c. The plurality of medical professionals 106a, 106b and 106c may access the professional interface for requesting certain number of blocks based on number of pending medical procedures (e.g., surgery cases) to be performed by them. In at least one example embodiment, the plurality of medical professionals 106a, 106b and 106c may release one or more blocks allocated to them if they do not need the block for any given day of week or a whole week using the facility management application 118. The block length of the one or more blocks released by a medical professional (e.g., the medical professional 106a) constitutes a released time of the medical professional. For example, if a medical professional 106b assigned with four blocks/week takes a break to go on a vacation, then the medical professional 106b may release the blocks (4 blocks) using the professional interface. If a medical professional (e.g., the medical professional 106b) releases too many blocks frequently, such blocks may be well-utilized by some other medical professional, say, medical professional 106c who may be in need of more blocks (or allocated time) for performing medical procedures. The devices 108a, 108b and 108c are exemplarily depicted as mobile phones. It is to be understood that the devices 108a, 108b and 108c associated with the medical professionals 106a, 106b and 106c, respectively, may be embodied in various forms, such as a tablet computer, a wearable device, a personal digital assistant, a laptop, and the like. The electronic devices 108a, 108b and 108c may be capable of being connected to the wireless communication network (such as the network 116).

A medical professional (e.g., the medical professional 106a) may access the facility 110 to perform medical procedures based on a facility scheduler table that indicates the details of the one or more blocks allocated to them. The environment 100 depicts the operating room 110 to be equipped with an electronic device 112 that stores the facility scheduler table comprising block schedule information of the medical professional 106a, 106b, 106c and also maintains OR case data of the medical procedures performed in the operating room 110. A medical professional (e.g., the medical professional 106b) performing a medical procedure logs in to the electronic device 112 using his/her login credentials to enter surgery details related with the medical procedure performed by him. The surgery details include log in and log out information of the medical professional 106b, patient wheel in and patient wheel out information, time taken to perform the medical procedure, type of medical procedure, patient information etc. The details may be stored in an electronic medical record (EMR) maintained at the electronic device 112.

In at least one example embodiment, the server 114 provides a software application, herein referred to as the facility management application 118, in response to a user request received from the user device 104 or the devices 108a, 108b, 108c (associated with the medical professionals 106a, 106b, 106c, respectively) via the network 116. Examples of the network 116 include stand alone or a combination of a local area network (LAN), a wide area network (WAN), wireless, wired, any currently existing or to be developed network that can be used for communication. More specifically, an example of the network 116 can be the Internet which may be a combination of a plurality of networks. The server 114 is configured to collect various forms of data, such as case scheduling data, facility scheduler table and surgery details of the medical professionals 106a, 106b, 106c from the electronic device 112 present in the operating room 110. The server 114 analyzes the various forms of data to determine facility usage data of the facility 110 by each medical professional of the plurality of medical professionals 106a, 106b and 106c. The facility usage data of each medical professional (e.g., the medical professional 106a) includes information about the allocated time of the medical professional, an actual usage time of the facility 110, an idle time of the facility 110 and a released time from the allocated time of the medical professional 106a. The ‘actual usage time’ represents time duration for which the medical professional 106a actually used the facility 110 for performing medical procedures, ‘idle time’ represents time duration for which the operating room 110 is not utilized despite being allocated to the medical professional (e.g., the medical professional 106a) for performing medical procedures and ‘released time’ represents a part of allocated time in which the medical professional releases at least a part of the allocated time of the operating room 110 allocated for the medical professional. For example, assuming the allocated time of the medical professional 106c is 15 hours/week or 5 blocks/week (B1, B2, B3, B4 and B5 of 3 hours each) of which the medical professional 106c uses 3 hours of the block B1 for performing a medical procedure, 1 hour of block B2 for performing a medical procedure, 2 hours and 30 minutes of block B3 for performing some medical procedure and releases the blocks B4, B5. As seen from the this example, the actual usage time of the facility 110 by the medical professional 106c is 6 hours and 30 minutes, an idle time of the facility 110 is 2 hours and 30 minutes as left unutilized by the medical professional 106c from the allocated time and released time of the facility 100 by the medical professional is 6 hours.

In at least one example embodiment, the facility management application 118 may provide an option for the user 102 to provide user inputs for determining a collectable time of each of the medical professionals 106a, 106b and 106c. The term ‘collectable time’ refers to at least a part of an unutilized time of the facility 100 from the allocated time for a medical professional (e.g., the medical professional 106c) that may be allocated to a different medical professional (e.g., the medical professional 106b). The user inputs comprise a first threshold and a second threshold. The first threshold is compared with the idle time of the facility to determine a collectable idle time. In at least one example embodiment, the idle time above the first threshold constitutes a collectable idle time. For example, if the medical professional 106c uses 1 hour (out of 3 hours) of block B2 for performing a medical procedure and 2 hours and 30 minutes (out of 3 hours) of block B3 for performing some medical procedure at the facility 100, the idle time of the facility 100 from the allocated time of the medical professional 106c is 2 hours of block B2 and 30 minutes of block B3. If the user 102 sets the first threshold for idle time as 2 hours, the idle time of 2 hours within the block B2 constitutes a collectable time whereas the idle time of 30 minutes within the block B3 does not constitute a collectable time.

The second threshold is compared with the released time to determine a collectable released time. In an embodiment, the released time above the second threshold constitutes the collectable released time from the allocated time of the medical professional. As illustrated by the above example, blocks B4 and B5 of block lengths of 3 hours have been released by the medical professional 106c, say for example, a week in prior to schedule of the blocks B4 and B5. In an example, the user 102 may provide the second threshold as 10% indicating that the medical professional 106c can release up to 10% of the one or more blocks allocated to him/her in a year. In such cases, if 5 blocks are allocated in a week, then a total of 320 blocks are allocated for the medical professional 106c in a year of which he/she can release 10% (or 32 blocks). Assuming, the medical professional 106c has already released 35 blocks, blocks B4 and B5 released by the medical professional 106c may exceed the threshold of 10% and constitute collectable blocks. The block length of the blocks B4 and B5 together constitute collectable released time of the medical professional 106c using the facility 100.

The collectable idle time and the collectable released time are aggregated to determine the collectable time of the medical professionals 106a, 106b and 106c using the facility 100. It shall be noted that the idle time below the first threshold and the released time below the second threshold constitute non-collectable time of the medical professionals 106a, 106b, 106c using the facility 100 and are not used to determine a number of collectable blocks from the medical professionals 106a, 106b, 106c using the facility 100. Further, the collectable time and the non-collectable time may vary based on the user inputs provided by the administrator.

For example, if collectable idle time of a medical professional (e.g., the medical professional 106c) is determined as 2 hours/week and the collectable released time is found to be 6 hours/week, then collectable time of the medical professional 106c using the facility 100 will be 8 hours/week (2 hours collectable idle time and 6 hours collectable released time). Further, the facility management application 118 is also configured to calculate a total number of collectable blocks from each medical professional of the plurality of medical professionals 106a, 106b and 106c. The total number of collectable blocks are based on the collectable time of the medical professional. The total number of collectable blocks is the ratio of the collectable time and a block length. For example, if block length is decided to be 2 hours and the collectable time is found to be 4 hours/week, then the collectable blocks will amount to 2 blocks/week (collectable time/block length=4/2).

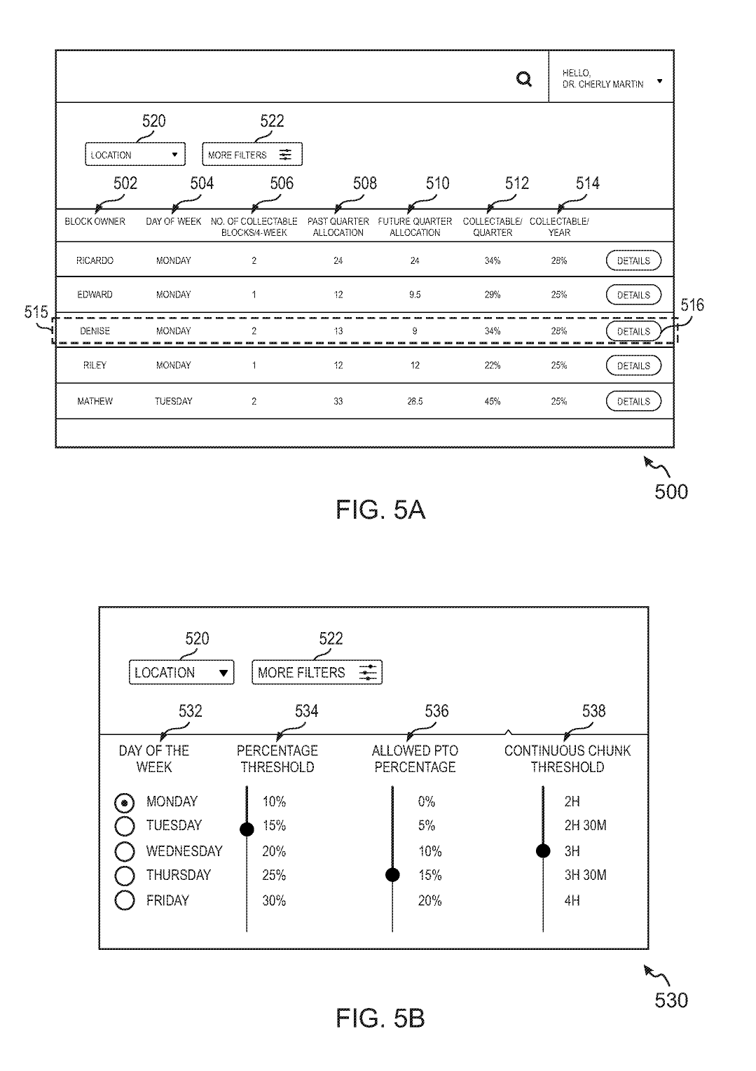

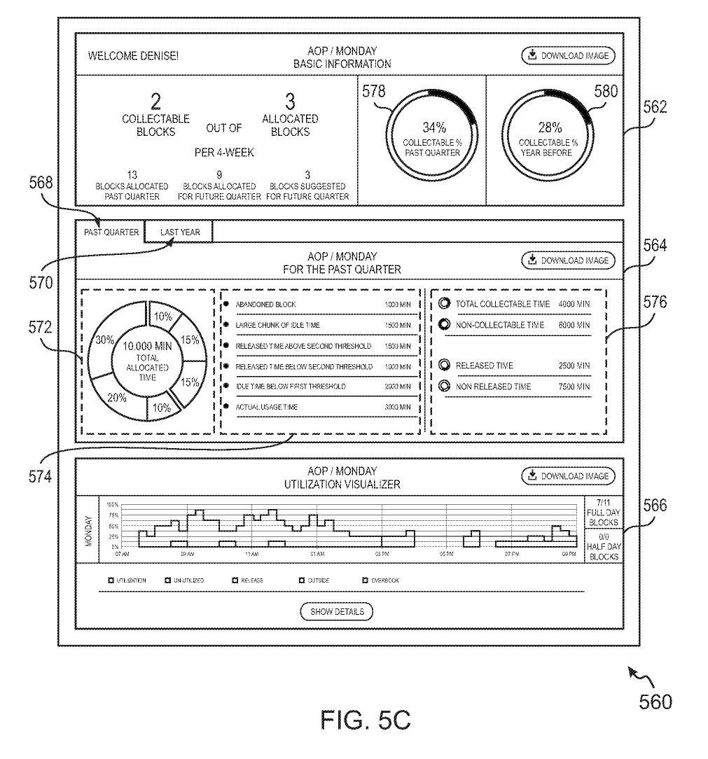

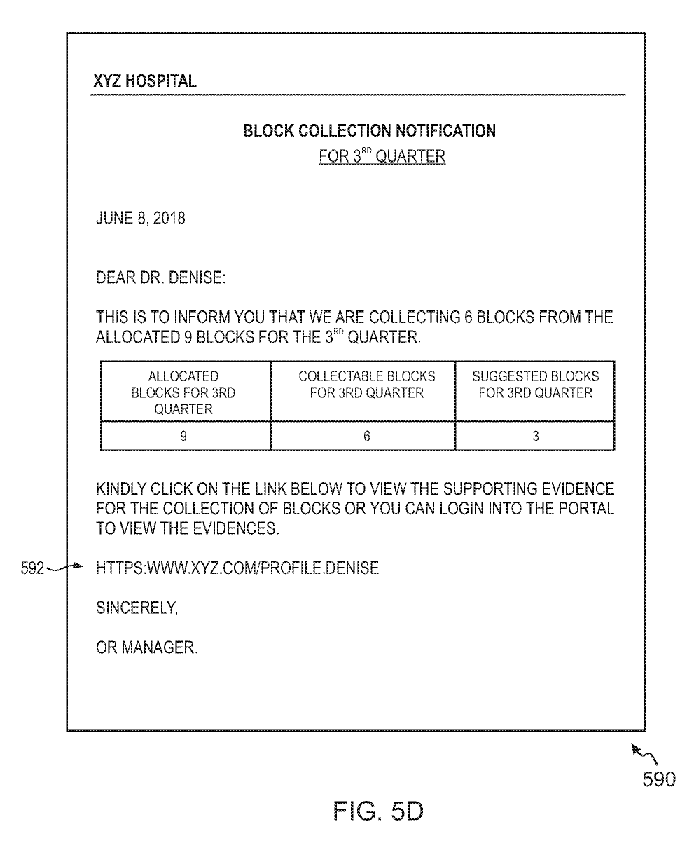

In at least one example embodiment, each medical professional (e.g., the medical professional 106a) can log in the professional interface of the facility management application 118 on a respective device (i.e. the device 108a) to view the block usage data of the medical professional using the facility 100. In an embodiment, the facility management application 118 may also be configured to display a supporting evidence for the total number of collectable blocks of the medical professional based on block usage data of the medical professional. The supporting evidence includes a total number of allocated blocks (based on the allocated time), a total number of collectable blocks, a non-collectable time and one or more data visualizations depicting the actual usage time, the idle time below the first threshold, the idle time above the first threshold, the released time below the second threshold and the released time above the second threshold. The non-collectable time of the medical professional is an aggregation of the actual usage time, the idle time below the first threshold and the release time below the second threshold. The UP s for block usage data and supporting evidence are explained with reference to FIGS. 5A and 5C.

The facility management application 118 may be an application/tool resting at the server 114. In an embodiment, the server 114 is configured to host and manage the facility management application 118 and communicate with user devices, such as the device 104, device 112 and the devices 108a, 108b and 108c. In an example, the user device 104 and the electronic devices 108a, 108b and 108c may access an instance of the facility management application 118 from the server 114 for installing on the device 104, device 112 and the devices 108a, 108b and 108c using application stores associated with Apple iOS™, Android™ OS, Google Chrome OS, Symbian OS®, Windows Mobile® OS, Windows Phone, BlackBerry® OS, Embedded Linux, web OS, Palm OS® or Palm Web OS™, and the like. Alternatively, the facility management application 118 may be accessed through the web using Internet.

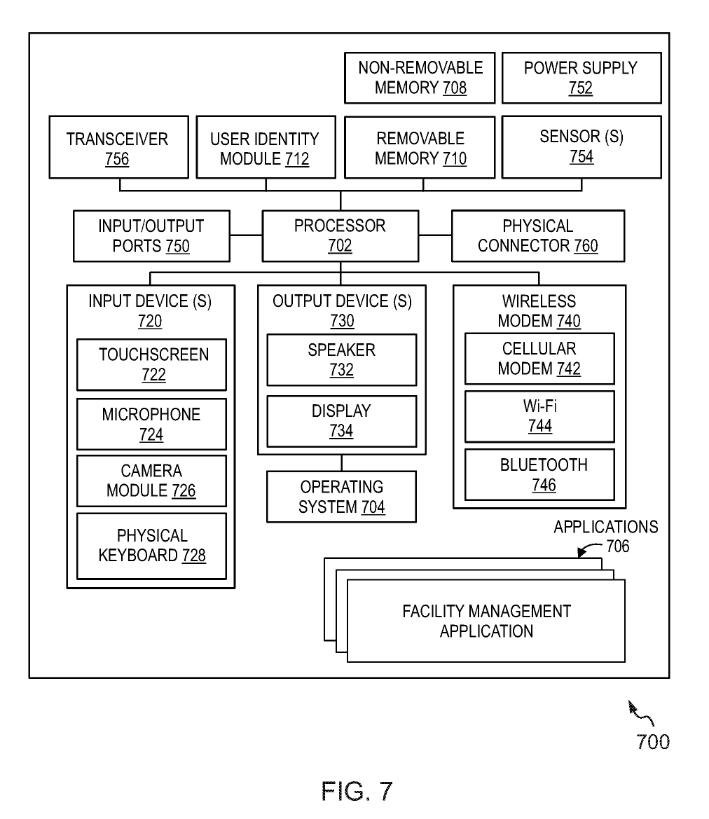

It is noted that the instructions (or the executable code) configuring the facility management application 118 are stored in a memory of the user device 104 after installation, and the instructions are executed by a processing module (for example, a single-core or a multi-core processor) included within the electronic device 104, as is exemplarily shown with reference to FIG. 7. Accordingly, even though the various functionalities for determining collectable time are explained with reference to or being performed by the facility management application 118, it is understood that the processing module in conjunction with the code in the memory is configured to execute the various tasks as enabled by the instructions of the facility management application 118.

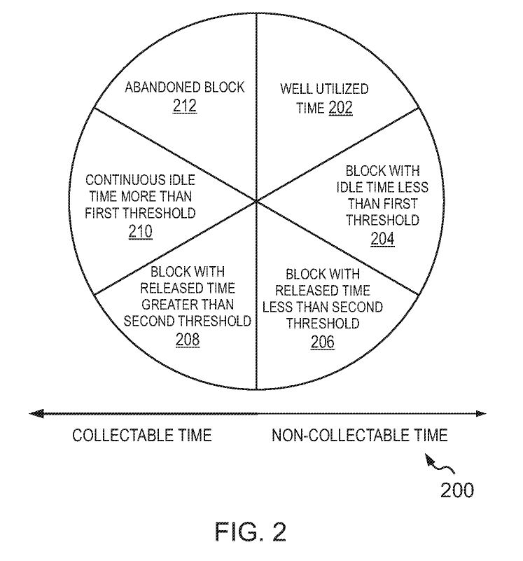

The facility usage data of the facility 100 by a medical professional (e.g., the medical professional 106a) is further explained in detail with reference to FIG. 2.

FIG. 2 is a graphical representation 200 depicting distribution of block usage data, in accordance with an embodiment. The graphical representation 200 depicts usage pattern of allocated blocks by a medical professional (e.g., the medical professional 106a) using a facility, for example, the operating room 110. It shall be noted that a cumulative block length of all allocated blocks of the medical professional for a predefined time (e.g., past quarter) is referred to as allocated time. The allocated time is broken into numerical proportions (or portions) based on a facility usage data of the facility by the medical professional.

The medical professional is scheduled to use the facility for performing medical procedures at the allocated time. For instance, the medical professional may be allocated 7 blocks (blocks B1, B2, B3, B4, B5, B6 and B7) in a week with different block lengths, say, block B1 of 3 hours, B2 of 2 hours, B3 of 4 hours, B4 of 2 hours, B5 of 1 hour, B6 of 3 hours and B7 of 4 hours for using the facility. The allocated time for the medical professional for using the facility is 19 hours/week. It shall be noted that the block length may be fixed or variable. The block usage data of the medical professional is shown on a circular statistical graph (graph representation 200). In this example representation, arc length of all slices of the graph 200 are shown to be equidistant for representation purposes only. However, it should be understood by a person skilled in the art that arc length of each slice may vary based on block usage data of each medical professional.

As seen in FIG. 2, the allocated blocks of the medical professional are divided into ‘well utilized time 202’, ‘idle time less than a first threshold 204’, ‘continuous idle time more than first threshold 210’, ‘blocks with released time less than second threshold 206’, ‘blocks with released time greater than second threshold 208’ and ‘abandoned blocks 212’. The ‘well utilized time 202’ represents block time utilized by the medical professional for performing the medical procedure. For example, if any portion of the block B2 of block length 2 hours is dedicatedly used by the medical professional, then the portion of time used will aggregate in this category. It shall be noted that time duration of the well-utilized block constitute non-collectable time.

The ‘idle time less than a first threshold 204’ represent a number of pockets/slots of idle time less than the first threshold set by the user in a block. For instance, the medical professional might have vacated the facility earlier than during time duration of the allocated block and an idle time of the block refers to unutilized time of the facility. For example, assuming the first threshold is set at 2 hours by an administrator (e.g., the user 102) and block B3 has been used for 1 hour and 40 minutes and block B1 has been used for 2 hours and 30 minutes, the block B3 has an idle time of 2 hours and 20 minutes (large chunk of unused time) greater than the first threshold (2 hours) and this idle time portion constitutes collectable time whereas block B1 has an idle time of 1 hour (small chunk of unused time) that does not constitute to collectable time (i.e. a non-collectable time).

The ‘blocks with released time less than second threshold 206’ represents number of blocks whose released time is less than second threshold set by the user. For instance, the medical professional might have released the block allocated to him for using the facility within a specific limit set for releasing blocks allocated to the medical professional. For instance, if the second threshold is set as 5%, it indicates that the medical professional can release allocated blocks upto 5%. The ‘blocks with released time greater than a second threshold 208’ represent number of blocks whose released time is greater than the second threshold set by the user. For instance, the medical professional might have released one or more blocks of the allocated blocks to him/her for using the facility after the specific limit (i.e. a predefined value) for releasing blocks. In an example, if the medical professional has been allocated 25 blocks per quarter and the second threshold is 5%, it implies that the medical professional can release upto one block/quarter. If the medical professional releases any block above the second threshold, say, 3 blocks of the allocated blocks in a quarter, then 2 blocks that were released beyond an allowed percentage (as indicated by the second threshold) accounts for collectable blocks from the medical professional.

The ‘abandoned blocks 212’ represent a number of blocks that are have not been used by the medical professional. For instance, if the medical professional is overloaded with out-patient consultation when he/she has to perform a medical procedure, for example, a block B5 (an allocated block) of 1 hour on Wednesday is left unutilized during a past quarter thereby resulting in the facility not being used efficiently. Such blocks are identified and constitute collectable blocks and a block length of the abandoned block 212 constitute the collectable time. In at least one example embodiment, the block length of abandoned blocks is greater than the first threshold or ideally the abandoned block refers to idle time of the facility that exceeds the first threshold. For example, abandoned blocks have a block length that constitutes idle time. Accordingly, abandoned blocks and blocks with large chunk of idle time (or idle time greater than the first threshold) constitute blocks with idle time greater than the first threshold.

As seen in the FIG. 2, the summation of number of ‘continuous idle time more than first threshold 210’, ‘blocks with released time greater than second threshold 208’ and ‘abandoned blocks 212’ account for the collectable time (avoidable unused time) of the medical professional. The collectable time may be calculated by adding up all pieces of collectable time. The collectable time accounts for time that may be taken from the medical professional and scheduled for other medical professionals so as to optimize utilization of the facility.

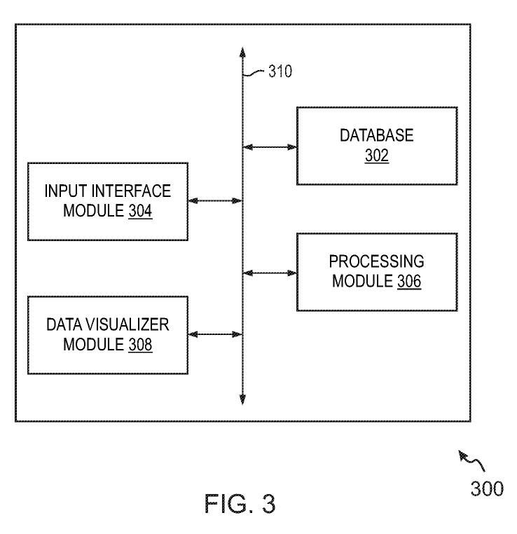

FIG. 3 is a block diagram representation of a system 300 for determining a collectable time associated with a medical professional using a facility, in accordance with an embodiment. The system 300 may be embodied in a server system, for example, the server 114 or an electronic device, such as, the electronic device 112 accessible by an administrator (e.g., the user 102) who manages the facility 100. The system 300 is configured to determine collectable time associated with each medical professional of a plurality of medical professionals (e.g., the medical professionals 106a, 106b and 106c). In an embodiment, the system 300 comprises a database 302, an input interface module 304, a processing module 306, a data visualizer module 308 and a centralized circuit system 310.

The database 302 is configured to store a facility usage data of each medical professional of the plurality of medical professionals working in the facility (e.g., the facility 100). The facility usage data comprises information related to an allocated time for the medical professional to use the facility, an actual usage time of the facility by the medical professional, an idle time of the facility from the allocated time and a released time of the facility by the medical professional from the allocated time. Further, the database is also configured to store EMRs. The EMR comprises OR case data, such as, information about patient wheel in and patient wheel out by the medical professional, medical procedure performed by the medical professional, equipment and medicines used during the medical procedure, time duration of the medical procedure, a log in and log out time of the medical professional and a facility scheduler table.

The input interface module 304 is configured to receive user inputs from the administrator for setting a first threshold and a second threshold. The value of the first threshold may be used to determine a collectable idle time and the value of the second threshold may be used to determine a collectable released time associated with each medical professional. In at least one example embodiment, the idle time below the first threshold and released time below the second threshold constitute a non-collectable time. The idle time above the first threshold and the released time above the second threshold constitute the collectable time of the medical professional. Examples of the input interface module 304 may include, but are not limited to, a keyboard, a mouse, a joystick, a keypad, a touch screen, soft keys, and the like.

The processing module 306 is in operative communication with the input interface module 304 and the database 302. The processing module 306 is configured to determine a collectable time of the medical professional using the facility based on the facility usage data and the second threshold for each medical professional. The collectable idle time is the idle time that is greater than the first threshold and the collectable released time is the released time that is greater than second threshold. After determining the collectable idle time and the collectable released time, the processing module 306 is configured to determine collectable time for each medical professional based on the collectable idle time and the collectable released time using formula:

Collectable time for each professional
= Collectable idle time associated with the professional
+ Collectable released time associated with the professional

The processing module 306 is further configured to determine a total number of collectable blocks based on the collectable time using the formula:

$$Number of collectable blocks = \frac{Collectable time}{Block length}$$

The block length may be fixed or variable. In some embodiments, the processing module 306 receives one or more block lengths via the input interface module 304 for determining the total number of collectable blocks from the medical professional. For example, if the collectable time of the medical professional is 10 hours, assuming, the administrator provides block lengths of 2 hours, 4 hours and 4 hours, the total number of collectable blocks are 3 blocks from the medical professional. In at least one example embodiment, a collectable percentage is determined by the processing module 306 for each medical professional of the plurality of medical professionals. The collectable percentage is a ratio of the total collectable time over the total allocated time multiplied by 100.

$$Collectable percentage = \frac{total collectable time}{total allocated time} * 100$$

The data visualizer module 308 is in operative communication with the processing module 306 and is configured to display one or more data visualizations for each medical professional based on a supporting evidence for the collectable time (or collectable blocks). In at least one example embodiment, the data visualizer module 308 in conjunction with the processing module 306 is configured to track the facility usage data of the medical professional and determine supporting evidence for the collectable time (or collectable percentage) of the medical professional using the facility. The supporting evidence includes at least one or more of a total number of allocated blocks based on the allocated time of the medical professional, a collectable time of the medical professional, a total number of collectable blocks based on block length and the collectable time, a non-collectable time, the block usage data of the medical professional comprising the actual usage time, the idle time below the first threshold, the idle time above the first threshold, the released time below the second threshold and the released time above the second threshold. In an embodiment, the non-collectable time is the cumulative of the actual usage time, the idle time below the first threshold and the release time below the second threshold.

In an embodiment, the processing module 306 is configured to generate a block decision letter for a medical professional based at least on the total number of collectable blocks, one or more data visualizations and the supporting evidence. The block decision letter may be sent to the medical professional for notifying him about the change in the allocated time. An example of the block decision letter sent to the medical professional is shown and explained with reference to FIG. 5D.

The database 302, the user interface module 304, the processing module 306, the data visualizer module 308 may be configured to communicate with each other via or through the centralized circuit system 310. The centralized circuit system 310 may be various devices configured to, among other things, provide or enable communication between the engines (302-308) of the system 300. In certain embodiments, the centralized circuit system 310 may be a central printed circuit board (PCB) such as a motherboard, a main board, a system board, or a logic board. The centralized circuit system 310 may also, or alternatively, include other printed circuit assemblies (PCAs) or communication channel media. In some embodiments, the centralized circuit system 310 may include appropriate storage interfaces to facilitate communication among the engines (302-308). Some examples of the storage interface may include, for example, an Advanced Technology Attachment (ATA) adapter, a Serial ATA (SATA) adapter, a Small Computer System Interface (SCSI) adapter, a RAID controller, a SAN adapter or a network adapter.

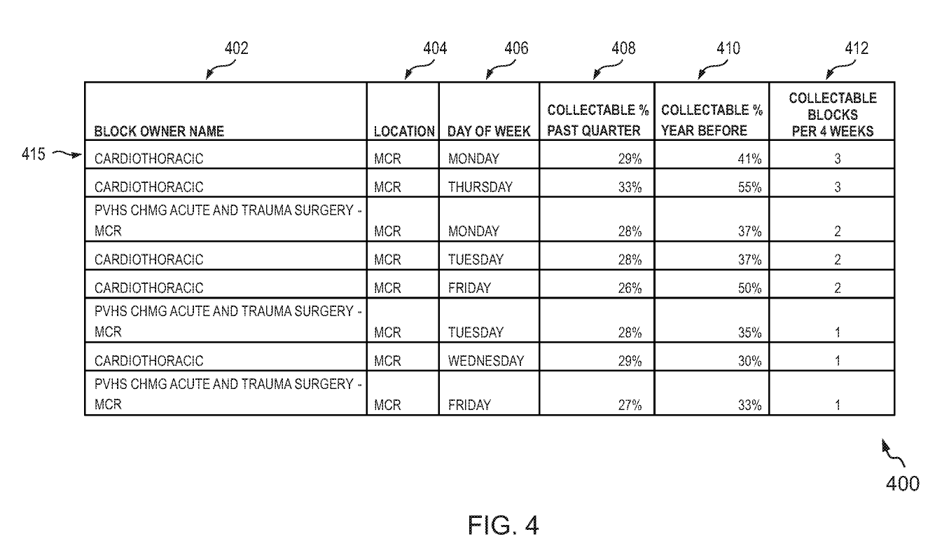

FIG. 4 is an example representation of a table 400 displaying collectable percentage and collectable blocks of each medical department, in accordance with an example embodiment. In at least one example embodiment, a medical department of a hospital is scheduled to use one or more allocated blocks (based on an allocated time for the medical department) of the facility for performing a medical procedure. It shall be noted that in such cases one or more medical professionals of the medical department may utilize the facility to perform the medical procedure. For example, if the facility is allocated to a cardiology department of the hospital, 2 medical professionals of the cardiology department may work in tandem on a patient for performing an angioplasty surgery. In such cases, each medical department may employ a department manager who may assign OR case data and decide the one or more medical professionals for performing medical procedures during a specific allocated time or allocated block for the medical department. As explained with reference to FIG. 2, a block usage data of the medical department is tracked for determining an actual usage time of the facility by the medical department from the allocated time, an idle time of the facility of the allocated time for the medical department and a released time of the facility of the allocated time for the medical department. The block usage data of the medical department is used to determine a collectable time and non-collectable time of the medical department.

The table 400 includes a block owner name field 402, a location field 404, a day of the week field 406, a collectable percentage past quarter field 408, a collectable percentage year before field 410 and collectable blocks per 4 weeks field 412. The field 402 indicates name of the medical department for which the block is allocated (allocated block) in a specific day of the week (as shown by the field 406). The location field 404 represents a facility/operating room assigned to the medical department. As already explained, the field 406 represents a day of the week for which blocks are assigned to the medical department for performing medical procedures. The field 408 displays a percentage of collectable blocks from the medical department over a past quarter and the field 410 (synonymous with the field 408) displays a percentage of collectable blocks from the medical department over a past year. The collectable percentage as shown for a medical department in the fields 408 and 410 may be used to determine a total number of collectable blocks of the medical department.

The field 312 indicates a total number of blocks that may be collected from the medical department based on the collectable percentage determined for the past quarter (shown by the field 408) and past year (shown by the field 410). A row 415 represents a medical department “Cardiothoracic” using the facility “MCR” on a day of the week “Monday” has a collectable percentage for past quarter as 29% and a collectable percentage year before as 41% indicating total number of collectable blocks from the Cardiothoracic department as “3 blocks/4 weeks”.

The facility management application 118 provisions one or more UIs for (1) acquiring a first threshold and a second threshold for determining the collectable idle time and the collectable released time, (2) displaying total number of collectable blocks in a predefined time, and (3) displaying supporting evidence for the total number of collectable blocks. The facility management application 118 is further explained with reference to FIGS. 5A-5D.

Referring now to FIG. 5A, an example representation of a UI 500 depicting data visualization for a plurality of medical professionals using the facility is shown in accordance with an example embodiment. The UI 500 is presented to an administrator (e.g., the administrator 102) who manages a facility such as, the facility 100. Further, the UI 500 may be displayed in response to user inputs provided by the administrator in the facility management application (shown in FIG. 5B).

The UI 500 displays a table for visualizing data associated with a plurality of medical professionals using the facility for performing medical procedures. The table includes a block owner field 502, a day of week field 504, No. of collectable blocks/4 weeks field 506, a past quarter allocation field 508, a future quarter allocation field 510, a collectable/quarter field 512 and a collectable/year field 514. It shall be noted that the table has been shown for exemplary purposes and the table may include fewer or more entries than those depicted in FIG. 5A.

A column associated with the field 502 lists names of the plurality of medical professionals whose data is being visualized by the administrator. The field 504 indicates the day of the week, for example, Tuesday for which collectable data of the plurality of medical professionals is visualized. The number of collectable blocks (e.g., 2 blocks) of each of the medical professional for that particular day of week in a four-week time frame is listed under the field 506. The number of blocks (e.g., 24 blocks) allocated to each medical professional of the plurality of medical professionals in a previous quarter for certain day of week (as indicated by the field 504) is displayed under the field 508 and the number of blocks (e.g., 22 blocks) suggested for future quarter for the day of week (shown by field 504) is displayed under the field 510. The collectable percentage (e.g., 34%) for previous quarter the day of week (shown by field 504) is listed under the field 512 and collectable percentage (e.g., 28%) for previous year is listed under the header collectable year 514. The collectable percentage for the previous quarter and the collectable percentage for the previous year are determined based on total number of collectable blocks for the medical professional in the past quarter and the past year, respectively. Determining collectable time, collectable blocks and collectable percentage of a medical professional are explained with reference to FIGS. 1-4.

A row 515 of the table shows a block owner “Denise” has number of collectable blocks/4 week as “2 blocks” on a day of week “Monday”. The block owner “Denise” was allocated “13 blocks” in the past quarter and “9 blocks” are planned to be allocated to him for future quarter for maintaining his surgical volume. Further, the block owner “Denise” had a collectable percentage of 34% for the past quarter and a collectable percentage of 28% for the past year. The row 515 of the table includes a details tab 516. Clicking on the details tab 516 for the block owner ‘Denise’ will navigate the user to the UI 560 of the facility management application. The administrator can view a supporting evidence for the collectable blocks for the certain day of week (Monday) associated with the block owner ‘Denise’ by providing a selection input on the tab 516. The UI 560 has been explained in detail with reference to FIG. 5C. In at least one example embodiment, the collectable data listed under the table changes based on user inputs of the administrator. The UI 500 includes an option 520 associated with text ‘LOCATION’ and an option 522 associated with text ‘MORE FILTERS’ displayed above the table. The option 520 is also a drop-down menu displaying various locations, for example, facilities/operating room available in the hospital in which medical procedures are performed. The administrator can choose the facility/operating room for which the table needs to be displayed. Each medical professional performs medical procedures in the facility assigned to him. So, if the user selects a facility from the drop down, the table will only display data associated with medical professionals who had performed medical procedures in that particular facility. The option 522 will present an overlay 530 on the UI 500 of the facility management application. The UI 530 may provide options for the administrator for setting thresholds. The UI 530 has been explained in detail with reference to FIG. 5B.

Referring now to FIG. 5B, an example representation of an overlay 530 depicting user inputs provided by an administrator for determining collectable time of the plurality of medical professionals using the facility is illustrated in accordance with an example embodiment. The overlay 530 is displayed to the administrator when he/she provides a selection input on the option 522 of the UI 500. The overlay 520 provides options for the administrator to set thresholds for determining the collectable time

The overlay 530 displays an option 532 associated with text “A DAY OF WEEK”, an option 534 associated with text “PERCENTAGE THRESHOLD”, an option 536 associated with text “ALLOWED PAID TIME OFF (PTO) PERCENTAGE” and an option 538 associated with text “CONTINUOUS CHUNK THRESHOLD”. The options 532, 534, 536 and 538 are filters that can be adjusted to determine the collectable time of a medical professional (as shown in UI 500). The option 532 displays a list of working days in a week of a hospital on which a facility can be used. The user can select any day from the working days and the data for the selected working day will only be displayed in the table (shown in UI 500). For example, if the administrator selects ‘Monday’, then facility usage data of Monday only will be considered and the table for Monday only will be displayed in the UI 500 (see, FIG. 5A). The option 534 in form of a slider provides a range of percentage values, for example, 10%, 20%, 30%, etc. The slider can be adapted to select any percentage value and the table will display collectable data only for the medical professionals whose collectable percentage is found to be greater than the selected percentage value. The option 534 provides an option for the administrator to visualize the collectable data of medical professionals that exceed a permissible threshold as stipulated by the hospital.

The option 536 comprises a slider that can be used to select a second threshold (also referred to as ‘a PTO percentage’), for example, 0%, 10%, 20%. The slider can be adapted to select any percentage value from the list of ranges. It shall be noted that the option 536 corresponds to the second threshold of the user input for determining the collectable released time. The PTO percentage is the amount of time medical professional releases from their allocated block time. The released time greater than the allowed PTO percentage may be considered as collectable released time. For example, if the administrator selects 15% as allowed PTO percentage, the medical professional can release up to 15% of the total allocated blocks. However, any blocks released above 15% will be considered as collectable released time.

The option 538 in form of a slider enables an administrator to select a first threshold (referred to as ‘continuous chunk threshold’) for determining a collectable idle time of the plurality of medical professionals. In this example representation, the slider can be adapted to select any time between a range of 2 hrs to 4 hrs. The idle time greater than the first threshold (set by the slider of option 538) accounts for collectable idle time. As seen in FIG. 5B, the administrator has selected 3 hrs as the continuous chunk threshold, so any continuous idle time greater than 3 hrs will be considered as collectable idle time. It shall be noted that abandoned blocks (blocks that were not utilized) also account for collectable idle time regardless of whether it is above the first threshold or not.

Referring now to FIG. 5C, an example representation of a UI 560 displaying supporting evidence for the collectable blocks of a medical professional is illustrated in accordance with an example embodiment. The UI 560 is presented to the administrator when he/she provides a selection input on the tab 516 of UI 500. In at least one example embodiment, the UI 560 is an example of the professional interface presented to the medical professional on a device, for example, device 108a of the medical professional 106a. The UI 560 provides one or more data visualizations as supporting evidence for the collectable blocks of the medical professional at an instance of location (AOP).

The UI 560 comprises a basic information block 562, a detailed information block 564 and a utilization visualizer block 566. A left part of the basic information block 562 displays a number of collectable blocks per 4 weeks out of the allocated blocks of the block owner, for example, 2 collectable blocks out of 3 allocated blocks. The left part also displays additional information of block allocation such as, number of blocks allocated in past quarter, number of blocks allocated for future quarter and blocks suggested for future quarter. As seen in FIG. 5C, for the medical professional ‘Denise’ for the day of week ‘Monday’, the collectable blocks are found to be 2 out of the allocated 3 blocks per 4 weeks, 13 blocks were allocated during the past quarter, 9 blocks are allocated for future quarter, and 9 blocks have been suggested to be allocated for the medical professional by the facility management application.

The basic information block 562 also presents graphical representations 578, 580 at a right part of the block 562. The graphical representation 578 graphically depicts a collectable percentage of the medical professional for the past quarter and the graphical representation 580 depicts a collectable percentage of the medical professional for the past year. As seen in FIG. 5C, for the medical professional ‘Denise’ for the day of week ‘Monday’, the collectable percentage for the past quarter is graphically depicted as 34% (see, graphical representation 578) and the collectable percentage for the past year is graphically depicted as 28% (see, graphical representation 580).

The detailed information block 564 displays supporting evidence for the collectable blocks suggested by the facility management application. The detailed information block includes a past quarter tab 568 and a last year tab 570. The administrator can provide a selection input on the past quarter tab 568 to view facility usage data of the medical professional for the past quarter of a year (as shown by block 564). Alternatively, the administrator can provide a selection input on the last year tab 570 to view facility usage data of the medical professional for the past year. The facility usage data comprises block usage data by the medical professional of a total allocated time in the past quarter for the selected day (Monday). The detailed information block 584 includes a left portion 572, a mid portion 574 and a right portion 576. The left portion 592 displays a graphical representation of facility usage data of the facility by the medical professional “Denise” on Monday. The graph depicts a percentage of abandoned blocks, a idle time less than first threshold, idle time greater than first threshold, released time less than second threshold, released time greater than second threshold and well-utilized blocks with different colors. The mid portion displays the block usage data on Mondays by the medical professional for the past quarter.

As seen in FIG. 5C, the allocated time of 10000 minutes for the medical professional “Denise” to use the facility on Monday comprises abandoned blocks with time amounting to 1000 minutes, large chunk of idle time as 1500 minutes, released time above a second threshold as 1500, released time below second threshold as 1000 minutes, idle time less than a first threshold as 2000 minutes and actual usage time as 3000 minutes. The abandoned blocks (1000 minutes) and the large chunk of idle time (1500 minutes) amount to idle time greater than the first threshold (2500 minutes). The right portion 596 may display a total collectable time, a non-collectable time, a released time and a non-released time of the medical professional along with one or more graphical visualizations that provide supporting evidence. The total collectable time (4000 minutes) is an aggregation of idle time greater than the first threshold (2500 minutes) and released time above the second threshold (1500 minutes). The non-collectable time (6000 minutes) is an aggregation of released time below second threshold (1000 minutes), idle time less than the first threshold (2000 minutes) and actual usage time (3000 minutes). The released time (2500 minutes) is an aggregation of released time above the second threshold (1500 minutes) and the released time below the second threshold (1000 minutes). The non-released time (7500 minutes) is an aggregation of idle time greater than the first threshold (2500 minutes), idle time less than the first threshold (2000 minutes) and actual usage time (3000 minutes)

The utilization visualizer block 586 may display a graph showing facility usage by the medical professional on the selected day of the week. The graph is an aggregation of time usage versus allocated time on a day of the week.

FIG. 5D is a representation of a block decision letter 590 sent to a medical professional, in accordance with an example embodiment. The block decision letter 590 is generated by the system 300. The block decision letter 590 is sent to the medical professional for notifying him about the change in the allocated number of blocks. The block decision letter 590 may include information about the number of collectable blocks that may be taken from the total number of allocated blocks in the past quarter (or past year). The block decision letter 590 may also include a hyperlink 592 to redirect the medical professional to a supporting evidence page (shown in FIG. 5C) showing evidence for the number of collectable blocks associated with the medical professional.

As seen in FIG. 5D, the block allocation letter 590 sent to the medical professional ‘Denise’ informs him about the change in the block allocation. The block allocation letter 590 indicates to him that he has been allocated with only “3 blocks” for the next quarter as collectable blocks amount to “6 blocks” that will be collected from him from the total allocated blocks of “9 blocks” for the third quarter.

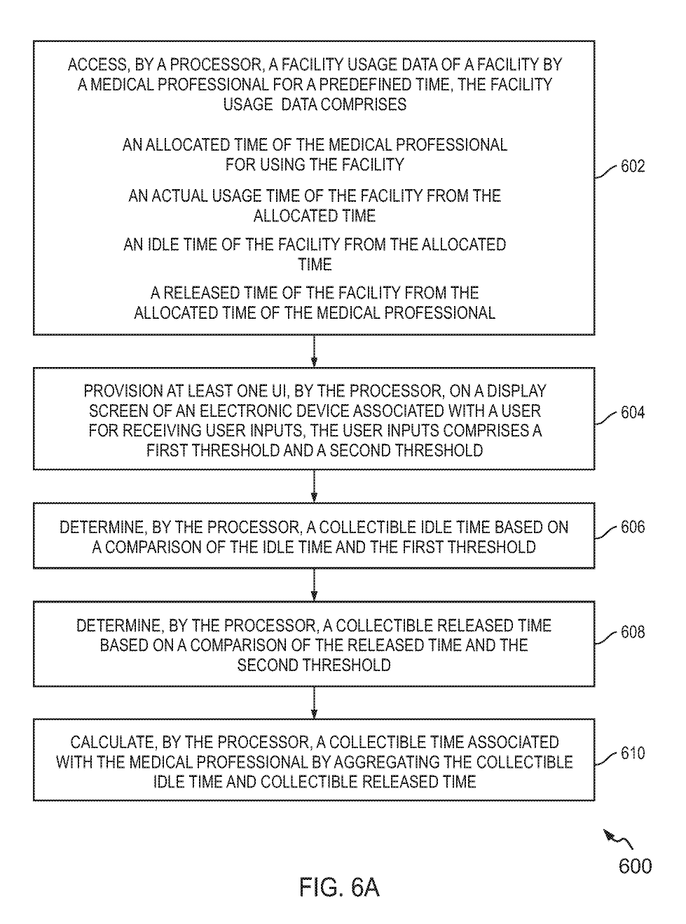

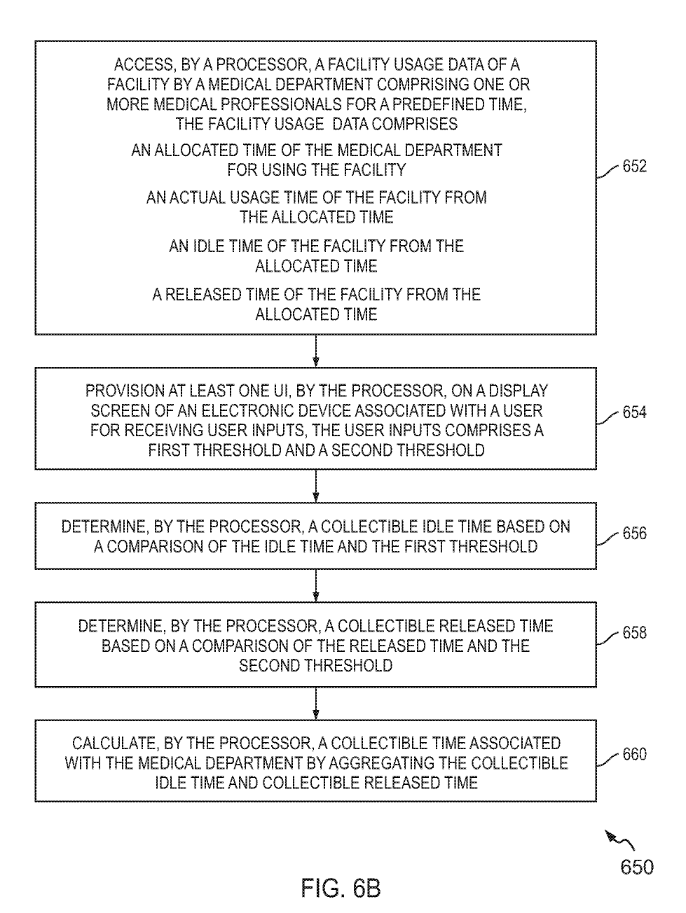

FIG. 6A is a flowchart illustrating a method 600 for determining collectable time of a medical professional using a facility, in accordance with an example embodiment. The operations of the method 600 may be carried out by a server such as the server 114, the system 300 or the electronic devices 104, 108a, 108b and 108c. The sequence of operations of the method 600 may not be necessarily executed in the same order as they are presented. Further, one or more operations may be grouped together and performed in form of a single step, or one operation may have several sub-steps that may be performed in parallel or in sequential manner.

At operation 602, the method 600 includes accessing, by a processor, a facility usage data of a facility by a medical professional for a predefined time. The facility usage data comprises an allocated time of the medical professional for using the facility, an actual usage time of the facility from the allocated time, an idle time of the facility from the allocated time and a released time from the allocated time of the medical professional. For example, the allocated time of the medical professional for a month is 10 hours and he/she has used it only for 3 hours and released 4 hours from the allocated 10 hours, then how the 10 hours are utilized by the medical professional is included in the facility usage data. The 10 hours of allocated time for using the facility is divided into the actual usage time which is the time during which the facility is actually used by the medical professional that is coming to be 3 hours, idle time is the time in which the facility is left unused in spite of being allocated to the medical professional for performing medical procedures which is coming 3 hours and released time is the part of allocated time in which the medical professional releases the facility allocated to him which is 4 hours. The predefined time can be any one of a block or a day or a month or a past quarter or a past year.

At operation 604, the method 600 includes provisioning at least one UI, by the processor, on a display screen of an electronic device associated with a user for receiving user inputs. The user input comprises a first threshold and a second threshold. The first threshold is associated with the idle time and the second threshold is associated with the released time associated with the medical professional for the predefined time.

At operation 606, the method 600 includes determining, by the processor, a collectable idle time based on a comparison of the idle time and the first threshold. The idle time determined at operation 602 is compared with the first threshold set by the user at operation 604 to determine a collectable idle time. In an embodiment, the collectable idle time corresponds to a single portion of continuous idle time which is greater than the first threshold. In an example, assuming that the first threshold is set to 1 hour, idle time is found to be 3 hours at an OR based on single portions of unused time at the OR, say 8:00 AM to 8:30 AM of a block B1 and a block B2 of unused time from 9:30 AM to 12:00 PM. The block B2 will aggregate (or constitute) collectable time of 2 hours 30 minutes as idle time is greater than the first threshold. However, the block B1 does not constitute a collectable time as idle time is less than the first threshold (1 hour). Therefore, of the idle time of 3 hours, the collectable time is 2 hours and 30 minutes (3 hours-30 minutes).

At operation 608, the method 600 includes determining, by the processor, a collectable released time based on a comparison of the released time and the second threshold. The released time determined at operation 602 is compared with the second threshold set by the user at operation 604 to determine collectable released time. In an embodiment, the collectable released time corresponds to the released time which is greater than the second threshold. For example, the medical professional can release upto 2 hours in a quarter that implies setting of the second threshold as 2 hours. If the medical professional has released 4 hours in the past quarter, then collectable released time will be 2 hours (4 hours-2 hours) from the released time of 4 hours.

At operation 610, the method 600 includes calculating, by the processor, a collectable time associated with the medical professional by aggregating the collectable idle time and the collectable released time. The collectable time is the avoidable waste time of each medical professional from the allocated time of using the facility i.e. the facility usage time that can be utilized by other medical professionals who are in need of facility usage time. The avoidable waste time of each medical professional is the summation of the collectable idle time and the collectable released time. For example, a medical professional's collectable idle time is found to be 2 hour/month and the collectable released time is found to be 2 hours/month, then his total collectable time will be 2+2 i.e. 4 hours/month.

FIG. 6B is a flowchart illustrating a method 650 for determining collectable time of a medical department using a facility, in accordance with an example embodiment. The operations of the method 650 may be carried out by a server such as the server 114, the system 300 or the electronic devices 104, 108a, 108b and 108c. The sequence of operations of the method 650 may not be necessarily executed in the same order as they are presented. Further, one or more operations may be grouped together and performed in form of a single step, or one operation may have several sub-steps that may be performed in parallel or in sequential manner.

At operation 652, the method 650 includes accessing, by a processor, a facility usage data of a facility by a medical department for a predefined time. The facility usage data comprises an allocated time of the medical department for using the facility, an actual usage time of the facility from the allocated time, an idle time of the facility from the allocated time and a released time of the facility from the allocated time of the medical department. For example, the allocated time of the medical department for a week is 10 hours and medical professionals working under that department has used it only for 3 hours and a department manager has released 4 hours from the allocated 10 hrs, then how these 10 hrs are utilized by the medical department is also included in the facility usage data. The 10 hrs of allocated time for using the facility can be divided into the actual usage time which is a time during which the facility is actually used by the medical professionals working under the medical department which is 3 hours, idle time is. the time in which the facility is left unused in spite of being allocated to the medical department for performing medical procedures which is coming 3 hrs and released time is the part of allocated time in which the medical department releases the facility allocated to him which is 4 hrs. In an embodiment, the predefined time is previous quarter. In an embodiment, the predefined time is previous year. The predefined time can be a block or a day or a month or a past quarter or a past year.

At operation 654, the method 650 includes provisioning at least one UI, by the processor, on a display screen of an electronic device associated with a user for receiving user inputs. The user input comprises a first threshold and a second threshold. The first threshold is associated with the idle time and the second threshold is associated with the released time associated with the medical department for the predefined time.

At operation 656, the method 650 includes determining, by the processor, a collectable idle time based on a comparison of the idle time and the first threshold. The idle time determined at operation 652 is compared with the first threshold set by the user at operation 654 to determine collectable idle time. In an embodiment, the collectable idle time corresponds to the idle time which is greater than the first threshold. For example, if the first threshold is set to 1 hour and the idle time found to be 3 hours, then collectable idle time for the week will be the idle time of 3 hours that is above the first threshold.

At operation 658, the method 600 includes determining, by the processor, a collectable released time based on a comparison of the released time and the second threshold. The released time determined at operation 652 is compared with the second threshold set by the user at operation 654 to determine collectable released time. In an embodiment, the collectable released time corresponds to the released time which is greater than the second threshold. For example, if the second threshold is set to 2 hours and the released time found to be 4 hours, then collectable released time for the week will be 4-2 i.e. 2 hours from the released time of 4 hours.

At operation 660, the method 650 includes calculating, by the processor, a collectable time associated with the medical department by aggregating the collectable idle time and the collectable released time. The collectable time is the avoidable waste time of the medical department from the allocated time of using the facility i.e. the usage time of facility that can be utilized by other medical departments who are requiring facility usage time. The avoidable waste time of the medical department is the summation of the collectable idle time and the collectable released time. For example, the medical department's collectable idle time is found to be 2 hour/week and the collectable released time is found to be 2 hours/week, then his total collectable time will be 2+2 i.e. 4 hours/week.

FIG. 7 shows a simplified block diagram of an electronic device 700 capable of implementing the various embodiments of the present disclosure. The electronic device 700 may be an example of the electronic devices 104, 108a, 108b and 108c. In an embodiment, the various operations related to determining collectable time of a medical professional or a medical department using a facility (e.g., the facility 100) can be facilitated using a facility management application installed in the electronic device 700. It should be understood that the electronic device 700 as illustrated and hereinafter described is merely illustrative of one type of device and should not be taken to limit the scope of the embodiments. As such, it should be appreciated that at least some of the components described below in connection with that the electronic device 700 may be optional and thus in an example embodiment may include more, less or different components than those described in connection with the example embodiment of the FIG. 7. As such, among other examples, the electronic device 700 could be any of a mobile electronic device or may be embodied in any of the electronic devices, for example, cellular phones, tablet computers, laptops, mobile computers, personal digital assistants (PDAs), mobile televisions, mobile digital assistants, or any combination of the aforementioned, and other types of communication or multimedia devices.

The illustrated electronic device 700 includes a controller or a processor 702 (e.g., a signal processor, microprocessor, ASIC, or other control and processing logic circuitry) for performing such tasks as signal coding, data processing, image processing, input/output processing, power control, and/or other functions. An operating system 704 control the allocation and usage of the components of the electronic device 700 and support for one or more applications programs (see, the facility management application 118) that implements one or more of the innovative features described herein. The applications 706 may include common mobile computing applications (e.g., telephony applications, email applications, calendars, contact managers, web browsers, messaging applications such as USSD messaging or SMS messaging or SIM Tool Kit (STK) application) or any other computing application. The touch-typing platform is configured to be in operative communication with other applications for example, through the OS or using API Calls, for enabling a user to learn the touch-typing skill.

The illustrated electronic device 700 includes one or more memory components, for example, a non-removable memory 708 and/or a removable memory 710. The non-removable memory 708 and/or the removable memory 710 may be collectively known as database in an embodiment. The non-removable memory 708 can include RAM, ROM, flash memory, a hard disk, or other well-known memory storage technologies. The removable memory 710 can include flash memory, smart cards, or a Subscriber Identity Module (SIM). The one or more memory components can be used for storing data and/or code for running the operating system 704 and the facility management application. The electronic device 700 may further include a user identity module (UIM) 712. The UIM 712 may be a memory device having a processor built in. The UIM 712 may include, for example, a subscriber identity module (SIM), a universal integrated circuit card (UICC), a universal subscriber identity module (USIM), a removable user identity module (R-UIM), or any other smart card. The UIM 712 typically stores information elements related to a mobile subscriber. The UIM 712 in form of the SIM card is well known in Global System for Mobile Communications (GSM) communication systems, Code Division Multiple Access (CDMA) systems, or with third-generation (3G) wireless communication protocols such as Universal Mobile Telecommunications System (UMTS), CDMA9000, wideband CDMA (WCDMA) and time division-synchronous CDMA (TD-SCDMA), or with fourth-generation (4G) wireless communication protocols such as LTE (Long-Term Evolution).

The electronic device 700 can support one or more input devices 720 and one or more output devices 730. Examples of the input devices 720 may include, but are not limited to, a touch screen/a display screen 722 (e.g., capable of capturing finger tap inputs, finger gesture inputs, multi-finger tap inputs, multi-finger gesture inputs, or keystroke inputs from a virtual keyboard or keypad), a microphone 724 (e.g., capable of capturing voice input), a camera module 726 (e.g., capable of capturing still picture images and/or video images) and a physical keyboard 728. Examples of the output devices 730 may include, but are not limited to a speaker 732 and a display 734. Other possible output devices can include piezoelectric or other haptic output devices. Some devices can serve more than one input/output function. For example, the touch screen 722 and the display 734 can be combined into a single input/output device.

A wireless modem 740 can be coupled to one or more antennas (not shown in the FIG. 7) and can support two-way communications between the processor 702 and external devices, as is well understood in the art. The wireless modem 740 is shown generically and can include, for example, a cellular modem 742 for communicating at long range with the mobile communication network, a Wi-Fi compatible modem 744 for communicating at short range with an external Bluetooth-equipped device or a local wireless data network or router, and/or a Bluetooth-compatible modem 746. The wireless modem 740 is typically configured for communication with one or more cellular networks, such as a GSM network for data and voice communications within a single cellular network, between cellular networks, or between the electronic device 700 and a public switched telephone network (PSTN).

The electronic device 700 can further include one or more input/output ports 750, a power supply 752, one or more sensors 754 for example, an accelerometer, a gyroscope, a compass, or an infrared proximity sensor for detecting the orientation or motion of the electronic device 700, a transceiver 756 (for wirelessly transmitting analog or digital signals) and/or a physical connector 760, which can be a USB port, IEEE 1294 (FireWire) port, and/or RS-232 port. The illustrated components are not required or all-inclusive, as any of the components shown can be deleted and other components can be added.

The disclosed systems and methods with reference to FIGS. 1 to 5, or one or more operations of the flow diagrams 600 and 650 may be implemented using software including computer-executable instructions stored on one or more computer-readable media (e.g., non-transitory computer-readable media, such as one or more optical media discs, volatile memory components (e.g., DRAM or SRAM), or non-volatile memory or storage components (e.g., hard drives or solid-state non-volatile memory components, such as Flash memory components) and executed on a computer (e.g., any suitable computer, such as a laptop computer, net book, Web book, tablet computing device, smart phone, or other mobile computing device). Such software may be executed, for example, on a single local computer or in a network environment (e.g., via the Internet, a wide-area network, a local-area network, a remote web-based server, a client-server network (such as a cloud computing network), or other such network) using one or more network computers. Additionally, any of the intermediate or final data created and used during implementation of the disclosed methods or systems may also be stored on one or more computer-readable media (e.g., non-transitory computer-readable media) and are considered to be within the scope of the disclosed technology. Furthermore, any of the software-based embodiments may be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

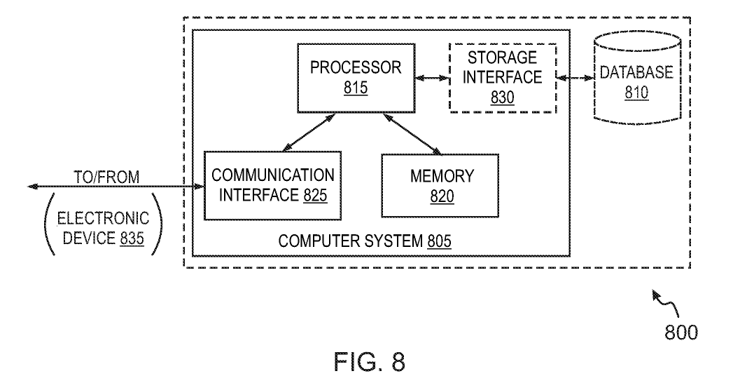

FIG. 8 is a simplified block diagram of a server system 800, in which the facility management application 118 may be stored, in accordance with one embodiment of the present disclosure. The server system 800 is an example of the server 114 shown and explained with reference to FIG. 1. The server system 800 includes a computer system 805 and one or more databases, such as a database 810.

The computer system 805 includes a processor 815 for executing instructions. Instructions may be stored in, for example, but not limited to, a memory 820. The processor 815 may include one or more processing units (e.g., in a multi-core configuration). The processor 815 is operatively coupled to a communication interface 825 such that the computer system 805 is capable of communicating with a remote device such as an electronic device 835. Some examples of the electronic device 835 may include, but are not limited to the electronic devices 104, 108a, 108b and 108c shown in FIG. 1.

The processor 815 may also be operatively coupled to the database 810. The database 810 is configured to store the facility management application 118 capable of determining collectable time associated with a medical professional or a medical department as explained with reference to FIGS. 1 to 6. The database 810 is any computer-operated hardware suitable for storing and/or retrieving data. The database 810 may include multiple storage units such as hard disks and/or solid-state disks in a redundant array of inexpensive disks (RAID) configuration. The database 810 may include, but not limited to, a storage area network (SAN) and/or a network attached storage (NAS) system.

In some embodiments, the database 810 is integrated within the computer system 805. For example, the computer system 805 may include one or more hard disk drives as the database 810. In other embodiments, the database 810 is external to the computer system 805 and may be accessed by the computer system 805 using a storage interface 830. The storage interface 830 is any component capable of providing the processor 815 with access to the database 810. The storage interface 830 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing the processor 815 with access to the database 810.

The memory 820 is a storage device embodied as one or more volatile memory devices, one or more non-volatile memory devices, and/or a combination of one or more volatile memory devices and non-volatile memory devices, for storing micro-contents information and instructions. The memory 820 may be embodied as magnetic storage devices (such as hard disk drives, floppy disks, magnetic tapes, etc.), optical magnetic storage devices (e.g., magneto-optical disks), CD-ROM (compact disc read only memory), CD-R (compact disc recordable), CD-R/W (compact disc rewritable), DVD (Digital Versatile Disc), BD (Blu-ray® Disc), and semiconductor memories (such as mask ROM, PROM (programmable ROM), EPROM (erasable PROM), flash ROM, RAM (random access memory), etc.).

Various example embodiments offer, among other benefits, techniques for establishing system and method for determining collectable time of a medical professional using a facility and thereby provides a unique set of visualizations that captures all necessary information efficiently and effectively for decision making, and thus easily determines block owners who has more avoidable time than others. A new block allocation system may be built using various features of the system. Such block allocation system may receive from requests for blocks from medical professionals and may allocate the collected blocks to the medical professionals who have already requested for blocks. Thereby, using technology of the present disclosure, effective utilization of the block time can be achieved.

Although the invention has been described with reference to specific exemplary embodiments, it is noted that various modifications and changes may be made to these embodiments without departing from the broad spirit and scope of the invention. For example, the various operations, blocks, etc., described herein may be enabled and operated using hardware circuitry (for example, complementary metal oxide semiconductor (CMOS) based logic circuitry), firmware, software and/or any combination of hardware, firmware, and/or software (for example, embodied in a machine-readable medium). For example, the apparatuses and methods may be embodied using transistors, logic gates, and electrical circuits (for example, application specific integrated circuit (ASIC) circuitry and/or in Digital Signal Processor (DSP) circuitry).

The present disclosure is described above with reference to block diagrams and flowchart illustrations of method and system embodying the present disclosure. It will be understood that various block of the block diagram and flowchart illustrations, and combinations of blocks in the block diagrams and flowchart illustrations, respectively, may be implemented by a set of computer program instructions. These set of instructions may be loaded onto a general-purpose computer, special purpose computer, or other programmable data processing apparatus to cause a device, such that the set of instructions when executed on the computer or other programmable data processing apparatus create a means for implementing the functions specified in the flowchart block or blocks. Although other means for implementing the functions including various combinations of hardware, firmware and software as described herein may also be employed.

Various embodiments described above may be implemented in software, hardware, application logic or a combination of software, hardware and application logic. The software, application logic and/or hardware may reside on at least one memory, at least one processor, an apparatus or, a non-transitory computer program product. In an example embodiment, the application logic, software or an instruction set is maintained on any one of various conventional computer-readable media. In the context of this document, a “computer-readable medium” may be any non-transitory media or means that can contain, store, communicate, propagate or transport the instructions for use by or in connection with an instruction execution system, apparatus, or device, such as a computer. A computer-readable medium may comprise a computer-readable storage medium that may be any media or means that can contain or store the instructions for use by or in connection with an instruction execution system, apparatus, or device, such as a computer.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omissions and substitutions of equivalents are contemplated as circumstance may suggest or render expedient, but such are intended to cover the application and\or implementation without departing from the spirit or scope of the claims.

What is claimed is:

1. A method, comprising:
accessing, by a processor, a facility usage data of a facility by a medical professional for a predefined time, the facility usage data comprising:
an allocated time of the medical professional for using the facility;
an actual usage time of the facility from the allocated time;
an idle time of the facility from the allocated time; and
a released time from the allocated time of the medical professional;
provisioning, by the processor, of at least one User Interface (UI), by the processor, on a display screen of an electronic device associated with a user for receiving user inputs, the user inputs comprising a first threshold associated with the idle time and a second threshold associated with the released time;
determining, by the processor, a collectable idle time based on a comparison of the idle time and the first threshold;
determining, by the processor, a collectable released time based on a comparison of the released time and the second threshold; and
calculating, by the processor, a collectable time associated with the medical professional by aggregating the collectable idle time and the collectable released time.

2. The method as claimed in claim 1, wherein determining the collectable idle time comprises:
determining, by the processor, a difference between the idle time of the medical professional and the first threshold for the allocated time, wherein the difference constitutes the collectable idle time of the medical professional.

3. The method as claimed in claim 1, further comprising:
receiving, by the processor, one or more block lengths from the user; and
determining, by the processor, a total number of collectable blocks from the medical professional based at least on the collectable time and the one or more block lengths.

4. The method as claimed in claim 3, further comprising:
displaying, by the processor, a supporting evidence for the collectable time of the medical professional for the predefined time, the supporting evidence comprising one or more of:
a total number of allocated blocks for the medical professional, wherein each allocated block is of a block length based on the allocated time;
a block usage data comprising the actual usage time, the idle time below the first threshold, the idle time above the first threshold, the released time below the second threshold and the released time above the second threshold;
the collectable time of the medical professional;
the total number of collectable blocks from the medical professional; and
a non-collectable time aggregating the actual usage time, the idle time below the first threshold and the release time below the second threshold.

5. The method as claimed in claim 4, further comprising:
displaying, by the processor, one or more data visualizations associated with the medical professional on the display screen, wherein the one or more data visualizations is based at least on the collectable time and the supporting evidence.

6. The method as claimed in claim 5, further comprising:
generating, by the processor, a block decision letter based at least on the total number of collectable blocks, the block decision letter comprising at least the supporting evidence.

7. The method as claimed in claim 1, wherein the predefined time is at least one of:
a block;
a day;
a month;
a past quarter; and
a past year.

8. A server, comprising:
a memory configured to store instructions; and
a processor configured to execute the stored instructions in the memory and thereby cause the server to perform:
accessing a facility usage data of a facility by a medical professional for a predefined time, the facility usage data comprising:
an allocated time of the medical professional for using the facility;
an actual usage time of the facility from the allocated time;
an idle time of the facility from the allocated time; and
a released time from the allocated time of the medical professional;
provisioning of at least one UI on a display screen of an electronic device associated with a user for receiving user inputs, the user inputs comprising a first threshold associated with the idle time and a second threshold associated with the released time;
determining a collectable idle time based on a comparison of the idle time and the first threshold;
determining a collectable released time based on a comparison of the released time and the second threshold; and
calculating a collectable time associated with the medical professional by aggregating the collectable idle time and the collectable released time.

9. The server as claimed in claim 8, wherein for determining the collectable idle time the server is caused to:
determine a difference between the idle time of the medical professional and the first threshold, wherein the difference constitutes the collectable idle time of the medical professional.

10. The server as claimed in claim 8, wherein the server is further caused to:
receive one or more block lengths from the user; and
determine a total number of collectable blocks from the medical professional based at least on the collectable time and the one or more block lengths.

11. The server as claimed in claim 10, wherein the server is further caused to:
display a supporting evidence for the collectable time of the medical professional for the predefined time, the supporting evidence comprising one or more of:
a total number of allocated blocks for the medical professional, wherein each allocated block is of a block length based on the allocated time;
a block usage data comprising the actual usage time, the idle time below the first threshold, the idle time above the first threshold, the released time below the second threshold and the released time above the second threshold;
the collectable time of the medical professional; and
the total number of collectable blocks from the medical professional; and
a non-collectable time aggregating the actual usage time, the idle time below the first threshold and the release time below the second threshold.

12. The server as claimed in claim 11, wherein the server is further caused to:
display one or more data visualizations associated of the medical professional based at least on the collectable time and the supporting evidence.

13. The server as claimed in claim 12, wherein the server is further caused to:
generate a block decision letter based at least on the total number of collectable blocks, the block decision letter comprises at least the supporting evidence.

14. A method, comprising:
accessing, by a processor, a facility usage data of a facility by a medical department comprising one or more medical professionals for a predefined time, the facility usage data comprising:
an allocated time of the medical department for using the facility;
an actual usage time of the facility from the allocated time;
an idle time of the facility from the allocated time; and
a released time from the allocated time of the medical department;
provisioning, by the processor, of at least one User Interface (UI), by the processor, on a display screen of an electronic device associated with a user for receiving user inputs, the user inputs comprising a first threshold associated with the idle time and a second threshold associated with the released time;
determining, by the processor, a collectable idle time based on a comparison of the idle time and the first threshold;
determining, by the processor, a collectable released time based on a comparison of the released time and the second threshold; and
calculating, by the processor, a collectable time associated with the medical department by aggregating the collectable idle time and the collectable released time.

15. The method as claimed in claim 14, further comprising:
determining, by the processor, a difference between the idle time of the medical department and the first threshold, wherein the difference constitutes the collectable idle time of the medical department.

16. The method as claimed in claim 14, further comprising:
receiving, by the processor, one or more block lengths from the user; and
determining, by the processor, a total number of collectable blocks from the medical department based at least on the collectable time and the one or more block lengths.

17. The method as claimed in claim 16, further comprising:
displaying, by the processor, a supporting evidence for the collectable time of the medical department for the predefined time, the supporting evidence comprising one or more of:
a total number of allocated blocks for the medical department, wherein each allocated block is of a block length based on the allocated time;
a block usage data comprising the actual usage time, the idle time below the first threshold, the idle time above the first threshold, the released time below the second threshold and the released time above the second threshold;
the collectable time of the medical department;
the total number of collectable blocks from the medical department; and
a non-collectable time aggregating the actual usage time, the idle time below the first threshold and the release time below the second threshold.

18. The method as claimed in claim 17, further comprising:
displaying, by the processor, one or more data visualizations for the medical department based at least on the collectable time and the supporting evidence.

19. The method as claimed in claim 18, further comprising:
generating, by the processor, a block decision letter based at least on the total number of collectable blocks and the supporting evidence.

20. The method as claimed in claim 14, wherein the predefined time is at least one of:
a block;
a day;
a month;
a past quarter; and
a past year.
